(19) United States
(12) Patent Application Publication
(10) Pub. No.: US 2023/0096889 A1
(43) Pub. Date: Mar. 30, 2023

(54) ELECTROSURGICAL RESECTOR TOOL

(71) Applicant: Creo Medical Limited, Chepstow (GB)

(72) Inventors: Christopher Paul Hancock, Chepstow (GB); Louis Turner, Chepstow (GB); Rohan Monico, Chepstow (GB); Malcolm White, Chepstow (GB); Simon Meadowcroft, Chepstow (GB); Steven Morris, Chepstow (GB); George Christain Ullrich, Bangor (GB)

(73) Assignee: Creo Medical Limited, Chepstow (GB)

(21) Appl. No.: 17/780,471

(22) PCT Filed: Nov. 24, 2020
(86) PCT No.: PCT/EP2020/083227

§ 371 Date: May 26, 2022

(30) Foreign Application Priority Data
Nov. 28, 2019 (GB) 1917324.4

(51) Int. Cl. A61B 18/14

(52) U.S. Cl. CPC A61B 18/1445; A61B 2018/00601

(57) ABSTRACT

Various embodiments provide an electrosurgical resector tool comprising: a shaft defining a lumen; an energy conveying structure for carrying electromagnetic (EM) energy through the lumen of the shaft; an instrument tip mounted at a distal end of the shaft. The instrument tip comprises: a static portion comprising a first blade element; and a movable portion comprising a second blade element, wherein the movable portion is movable relative to the static portion between a closed position in which the first blade element and second blade element lie alongside each other to an open position in which the second blade element is spaced from the first blade element by a gap for receiving biological tissue. The instrument tip also includes a travel limiting mechanism operable to limit a maximum extent of relative movement between the second blade element and the first blade element in the open position and/or the closed position. The instrument tip further includes a first electrode, a second electrode and a planar dielectric body, the first and second electrodes being spaced apart and electrically isolated from each other by the planar dielectric body, and wherein the first electrode and the second electrode are connected to the energy conveying structure for delivery of the EM energy from the instrument tip. The tool further comprises an actuator for controlling relative movement between the movable portion and the static portion.

### FIELD OF THE INVENTION

The invention relates to an electrosurgical resector tool, for cutting, coagulating and ablating biological tissue using electromagnetic (EM) energy. In particular, the invention relates to an electrosurgical resector tool having first and second blade elements which are movable relative to each other between open and closed positions, and further having a travel limiting mechanism operable to limit a maximum extent of relative movement between the first and second blade elements in the open position and/or the closed position.

### BACKGROUND TO THE INVENTION

Surgical resection is a means of removing sections of organs from within the human or animal body. The organs may be highly vascular. When tissue is cut (i.e. divided or transected), small blood vessels may be damaged or ruptured. Initial bleeding is followed by a coagulation cascade where the blood is turned into a clot in an attempt to plug the bleed. During an operation it is desirable for a patient to lose as little blood as possible, so various devices have been developed in an attempt to provide bleeding-free cutting. For endoscopic procedures, it is also undesirable for a bleed to occur and not to be dealt with expediently, since the flow of blood may obscure the operator's vision. Instead of a sharp blade, it is known to use RF energy to cut biological tissue. The method of cutting using RF energy operates using the principle that as an electric current passes through a tissue matrix (aided by the ionic cell contents), the impedance to electron flow across the tissue generates heat. When a pure sine wave is applied to the tissue matrix, enough heat is generated within the cells to vaporize the water content of the tissue. There is thus a huge rise in the internal cell pressure that cannot be controlled by the cell membrane, resulting in rupture of the cell. When this occurs over a large area, it can be seen that the tissue is transected.

The above procedure works elegantly in lean tissue, but it is less efficient in fatty tissue because there are fewer ionic constituents to aid the passage of electrons. This means that the energy required to vaporize the contents of the cells is much greater, since the latent heat of vaporization of fat is much greater than the latent heat of vaporization of water. RF coagulation operates by applying a less efficient waveform to the tissue, whereby instead of being vaporized, the cell contents are heated to around 65° C., drying out the tissue by desiccation and denaturing the proteins in the vessel walls. This denaturing acts as a stimulus to the coagulation cascade, so clotting is enhanced. At the same time the collagen in the wall is denatured, turning from a rod-shaped to a coil-shaped molecule, causing the vessel to contract and reduce in size, giving the clot an anchor point, and a smaller area to be plugged.

However, RF coagulation is less efficient when fatty tissue is present because the electrical effect is diminished. It can thus be very difficult to seal fatty bleeders. Instead of having clean white margins, the tissue has a blackened burned appearance.

### SUMMARY OF THE INVENTION

At its most general the present invention provides a development to the electrosurgical resector tool concept discussed in GB2567480. The electrosurgical resector tool has an energy delivery structure that facilitates biological tissue cutting and sealing using electromagnetic (EM) energy. In particular, the invention relates to combined actuation and energy delivery mechanisms that are compact enough to enable the tool to be insertable through an instrument channel of a surgical scoping device, such as an endoscope, gastroscope or bronchoscope. The device could also be used to perform laparoscopic or open surgery, i.e. the bloodless resection of a liver lobe with the abdominal cavity open.

The electrosurgical resector tool has an instrument tip having first and second blade elements which are movable relative to each other between open and closed positions, and the development may include a travel limiting mechanism operable to limit a maximum extent of relative movement between the first and second blade elements in the open and/or the closed positions. In this way, over-stressing the resector tool jaws can be avoided and smooth, predictable jaw movement can be ensured.

Additionally, the electrosurgical resector tool may include a control rod for controlling relative movement between the first and second blade elements, and the development may include a set of overlapping tubes which provide a channel through which the control rod can slide and which is fixed to the instrument tip. In this way, movement of the control rod can be smooth and predictable.

According to a first aspect of the present invention, there is provided an electrosurgical resector tool comprising: a shaft defining a lumen; an energy conveying structure for carrying electromagnetic (EM) energy through the lumen of the shaft; an instrument tip mounted at a distal end of the shaft, wherein the instrument tip comprises: a static portion comprising a first blade element; and a movable portion comprising a second blade element, wherein the movable portion is movable relative to the static portion between a closed position in which the first blade element and second blade element lie alongside each other to an open position in which the second blade element is spaced from the first blade element by a gap for receiving biological tissue; a travel limiting mechanism operable to limit a maximum extent of relative movement between the second blade element and the first blade element in the open position and/or the closed position; a first electrode, a second electrode and a planar dielectric body, the first and second electrodes being spaced apart and electrically isolated from each other by the planar dielectric body, and wherein the first electrode and the second electrode are connected to the energy conveying structure for delivery of the EM energy from the instrument tip; and an actuator for controlling relative movement between the movable portion and the static portion. The actuator may be a separate element to the instrument tip, but connected to the instrument tip in order to open and close the blade elements.

Optionally, one of the first blade element and the second blade element comprises the planar dielectric body extending longitudinally and having the first electrode on a first laterally facing surface thereof, and wherein, in the closed position, the other of the first blade element and the second blade element lies adjacent to a second laterally facing surface of the longitudinally extending planar dielectric body opposite to the first laterally facing surface thereof.

Optionally, the second blade element has a length commensurate with a length of the first blade element.

Optionally, the energy conveying structure comprises a coaxial transmission line extending in a longitudinal direction through the lumen. The coaxial transmission line comprises an inner conductor separated from an outer conductor by a dielectric material. The inner conductor is connected to one of the first electrode and the second electrode and the outer conductor is connected to the other of the first electrode and the second electrode, for delivery of the EM energy from the instrument tip.

Optionally, the energy conveying structure is for carrying radiofrequency (RF) electromagnetic (EM) energy and microwave EM energy, and wherein the first electrode and the second electrode are operable: as active and return electrodes for delivering RF energy conveyed from the energy conveying structure; and a microwave field emitting structure for delivering microwave energy conveyed from the energy conveying structure. The electrosurgical resector tool may provide a plurality of operational modalities that facilitate biological tissue cutting and sealing using radiofrequency (RF) electromagnetic energy and/or microwave EM energy. In one example, the electrosurgical resector tool may comprise a pair of blade elements that provide a scissor-like mechanism that can provide three complimentary modalities: (i) a gliding RF-based cut when the blade elements are closed, (ii) a scissor-type cut performed on tissue grasped between the blade elements using a combination of RF energy and applied pressure, and (iii) a coagulation or vessel sealing operation performed on tissue grasped between the blade elements using a combination of microwave energy and applied pressure. Moreover, the RF and/or microwave energy may be supplied in any of these modalities at a power level sufficient to cause tissue ablation. By suitable configuration of a pair of electrodes on the blade elements, the supplied RF or microwave energy in each of these operational modalities can be focussed in the region required. The pair of electrodes may be both on the same blade element, or there may be an electrode on each blade element. However, it is to be understood that in some embodiments, only RF EM energy, or only microwave EM energy may be delivered.

In this structure, the first and second blade elements may resemble a scissors-type closure mechanism. Thus, the second blade element may be arranged to slide past the first blade element during movement between the open position and closed position, e.g. to effect mechanical cutting through application of a shearing force. The movable portion may be movable relative to the static portion in a plane parallel to a plane defined by the planar dielectric body. Herein the term “static” may mean that fixed in relation to the distal end of the shaft when in use (i.e. when the second blade element is moved between the open and closed position).

The shaft may be flexible, e.g. suitable for bending or other steering to reach the treatment site. A flexible shaft may enable the device to be usable in a surgical scoping device such as an endoscope. In other examples, the shaft may be rigid, e.g. for use in open surgery or with a laparoscope.

The first electrode and second electrode may be disposed at the cutting interface. In one example, both electrodes are on the same blade element, which may be on either the movable portion or the static portion. For example, the second electrode may be located on the second laterally facing surface of the longitudinally extending planar dielectric body. This may assist in provide uniform energy delivery at the cutting interface. Where both electrodes are on one blade element, the other blade element may be electrically inert, e.g. made of plastic or other insulator.

In another example, the first electrode may be on one of the blade elements, and the second electrode on the other blade element. For example, the longitudinally extending planar dielectric body may be on the first blade element, and the second electrode may extend along a side of the second blade element.

The first and second electrodes may thus be disposed along each side of the cutting interface, with the planar dielectric body in between. In this arrangement, if RF EM energy is applied to the electrodes the RF EM energy flows preferentially between the first and second blade elements across the cutting interface. Similarly, if microwave EM energy is applied while the blade elements are open, a microwave field emitted by the electrodes has a much higher field strength within the gap between the blade elements than elsewhere.

When in the closed position, the second electrode is separated from the first electrode along much of its length by the planar dielectric body. If RF EM energy is applied in this position, the RF EM energy preferentially flows around a distal tip and side edge of the closed blade elements, which facilitates a RF-only gliding cut performed by sliding the instrument tip through tissue.

The movable portion and thus the second blade element may be formed from an insulator-coated conductive material which is further coated with parylene N. For example, the movable portion may be a cast piece of stainless steel having a ceramic (e.g. alumina spray), synthetic plastic (e.g. Bakelite), diamond-like carbon (DLC), enamel coating, or a silicon-based paint coating. The second electrode may be formed at a side portion of the second blade element where the insulator coating and the parylene N coating is removed. The second electrode may be the exposed conductive material of the movable portion, or may comprise an additional conductive layer (e.g. of gold or the like) deposited or otherwise affixed to the exposed conductive material.

The second blade element may comprise a laterally protruding flange along its side portion. The flange thus protrudes towards the first blade element when in the closed position. The second electrode may be formed on a laterally facing edge of the laterally protruding flange.

The travel limiting mechanism may be a feature of the instrument tip. As such, structural features of the instrument tip may cooperate to define the relative positions of the first and second blade elements in the open and/or closed positons. This results in open and/or closed positions which are consistent and do not vary between applications. This may be different to conventional techniques in which the actuator or control rod defines these relative positions by having a limited travel. That is, conventionally, the amount of distance the control rod can slide within the shaft may be limited, for example, by a handpiece at a proximal end of the shaft. Given the flex of the various elements in the shaft, this type of mechanism can result in a variable open position and/or closed position, which can be undesirable in certain precision operations that the instrument tip is used to perform. The travel limiting mechanism may be formed by one or more pairs of cooperating structures formed on the static portion and the movable portion. That is, for each pair, one cooperating structure is formed on the static portion and the other cooperating structure is formed on the movable portion.

One pair of cooperating structures may function to limit a maximum extent of relative movement between the second blade element and the first blade element in the open position, whereas another pair of cooperating structures may function to limit a maximum extent of relative movement between the second blade element and the first blade element in the closed position. The travel limiting mechanism may limit a maximum angle between the first and second blade elements in the open position to be about 60 degrees.

A first pair of cooperating structures may include a raised protrusion and a cooperating stop surface (which may be substantially flush with surrounding surfaces), wherein the raised protrusion and the stop surface are configured or arranged in use to abut each other in the open position. That is, moving the moveable portion into the open position moves the raised protrusion into contact with the stop surface such that further opening of the first and second blade elements is prevented. That is, the second blade element is prevented from moving further past the first blade element. The stop surface and/or the raised protrusion may be specially formed structures which are sized and/or shaped to limit how far apart the first and second blade elements can move. In an embodiment, the moveable portion comprises the raised protrusion and the static portion comprises the stop surface. Specifically, the raised protrusion may be formed on a top surface of the moveable portion and distally of a connection (e.g. pivotal connection) between the movable portion and the static portion. Also, the stop surface may be formed on a top surface of the static portion and proximally of the connection between the movable portion and the static portion. The stop surface may be provided by a slot formed in the static portion by a support arm to which the movable portion is attached.

A second pair of cooperating structures may include a pair of abutment surfaces, wherein the pair of abutment surfaces are configured in use to abut each other in parallel formation in the closed position. That is, moving the movable portion into the closed position moves the two abutment surfaces together such that they contact each other and are substantially parallel to each other. By contacting along a surface rather than a point, the tool can provide a strong and reliable closure mechanism which can be advantageous, for example, when severing tissue using the first and second blade elements. In an embodiment, a first abutment surface is formed on a top surface of the movable portion and proximally of a connection (e.g. pivotal connection) between the moveable portion and the static portion. The first abutment surface may be formed as the top surface of an attachment plate of the movable portion, wherein the attachment plate is a proximal extension of the movable portion that extends proximally of the connection to the static portion. The attachment plate may be sized and/or shaped to limit how far the second blade element can move past the first blade element in the closing direction (i.e. the direction of travel from the open position to the closed positon). Also, a second abutment surface is formed on an under surface of the static portion and proximally of the connection between the moveable portion and the static portion. The second abutment surface may be formed as an underside of a support arm of the static portion. The support arm may be a lateral and forward (i.e. distally extending) extension of the static portion which defines a slot to accommodate movement of the movable portion relative to the static portion. The moveable portion may be connected (e.g. pivotally connected) to the static portion by the support arm. The support arm may be sized and/or shaped to limit how far the second blade element can move past the first blade element in the closing direction (i.e. the direction of travel from the open position to the closed positon).

As mentioned, the static portion may comprise a support arm on which the movable portion is mounted, and the support arm may define a slot in the static portion for receiving part of the movable portion. A length of the slot (i.e. the dimension in line with the shaft length) may be between 1 mm and 3 mm (preferably less than about 2 mm). A width of the slot (i.e. the dimension in line with the pivot axis) may be between 0.2 mm and 1.2 mm (preferably more than about 0.7 mm). A depth of the slot may be between 0.2 mm and 1.2 mm (preferably more than about 0.6 mm). The slot may be necessary in order to provide space for part of the moveable portion (e.g. a proximal part) to move relative to the static portion between the open and closed positions. The support arm may form part of an electrical connection between the energy conveying structure and the second electrode. For example, the static portion (e.g. the support arm) may be formed from an insulator-coated conductive material which is further coated with parylene N, and may comprise a proximal contact portion at which the insulator coating and the parylene N coating is removed and which is electrically connected to the inner conductor or outer conductor of the coaxial transmission line. An advantage of limiting dimensions of the slot is that it is possible to ensure a higher quality coating (e.g. of insulator and/or parylene N). For example, it is easier to ensure that the coating is complete and even. The static portion (e.g. the support arm) may have a proximal recess for attachment to a distal end of the coaxial transmission line. Other types of electrical connection may also be used. For example, a flexible conductor may be connected between the energy conveying structure (e.g. the inner conductor or outer conductor of the coaxial transmission line) and the first electrode or second electrode. Preferably the length of any flexible conductor is equal to or less than an eighth of a wavelength of the microwave energy, in order to prevent it from affecting the emitted field.

The coaxial transmission line may be adapted to convey either of or both of RF EM energy and microwave EM energy. Alternatively, the energy conveying structure may comprise different routes for the RF EM energy and microwave EM energy. For example, the microwave EM energy may be delivered through the coaxial transmission line, whereas the RF EM energy can be delivered via twisted pair wires or the like. Where a separate energy delivery route is provided, the first and second electrodes may comprise separate RF electrode portions and microwave electrode portions to enable the RF energy and microwave energy to be delivered from different regions of the instrument tip. For example, the microwave energy may be delivered from one of the blade elements, whereas the RF energy may be delivered between the blade elements. In another embodiment, the electrosurgical tool is only configured to deliver only one of RF EM energy and microwave EM energy.

The movable portion may be mounted to the support arm via a pivot connection. For example, the support arm may provide a clevis-type structure that supports a pivot axle on which the movable portion is mounted. The electrical connection between the energy conveying structure and the second electrode may pass through the pivot connection. For example, the pivot axle may be formed from a conductive material, and the insulator coating (and the parylene N coating) of the movable portion and the support arm may be removed where they respectively contact the pivot axle.

The dielectric material and inner conductor of the coaxial transmission line may extend beyond a distal end of the outer conductor. The inner conductor may include an exposed distal portion that is electrically connected to the first electrode, e.g. by directly overlapping with and contacting a proximal portion of the first electrode.

The movement between the movable portion and the static portion may be rotational or translational or a combination of the two. In one example, the movable portion may be pivotable relative to the static portion, whereby the second blade element is angled relative to the first blade element in the open position. This example may resemble a conventional scissor-type closure. The second blade element may be movable through only an acute angle (i.e. not an obtuse angle) between the open position and the closed position. In an embodiment, the travel limiting mechanism may be configured to limit the acute angle to between 90 degrees and 40 degrees, and preferably between 80 degrees and 50 degrees, and more preferably about 60 degrees. Additionally or alternatively, the travel limiting mechanism may be configured to limit a maximum distance between the jaws in the open position to about 3.5 mm.

The actuator may comprise a control rod slidably mounted in the flexible shaft. The control rod may have an attachment feature engaged with the movable portion, whereby longitudinal movement of the control rod in the shaft causes movement of the movable portion relative to the static portion. The attachment feature may be a hook or any suitable engagement for transmitting push and pull forces to the movable portion. The movable portion may include an aperture (e.g. a circular hole) and the attachment feature (e.g. hook) may be configured to fit within the hole to drive movement of the second blade element past the first blade element. The circular hole diameter may be only slightly larger than the control rod diameter, so that the attachment feature (e.g. hook) is prevented from moving longitudinally inside the hole. This may ensure that the jaw movement is smooth and predictable since most or all control rod longitudinal sliding movement is translated into jaw movement.

The static portion may comprise a support arm that provides a mounting base (e.g. a pivot base) for the movable portion. The planar dielectric body may be a separate piece of material mounted on, e.g. adhered or otherwise affixed to, the support arm. The planar dielectric body may be formed from ceramic (e.g. alumina). Herein, reference to “planar” material may mean a flat piece of material having a thickness that is substantially less that its width and length. The planar dielectric body may have a length dimension aligned in the longitudinal direction, a thickness dimension aligned in a lateral direction, and a width dimension orthogonal to both the length and thickness dimensions. A plane of the planar dielectric body is that in which the length and width dimensions lie, i.e. a plane orthogonal to the width dimension.

The first electrode may be a conductive material (e.g. gold) deposited or otherwise mounted on the first laterally-facing surface of the planar dielectric body. The second laterally-facing surface of the planar dielectric body that faces in an opposite direction to the first laterally-facing surface may be exposed at the cutting interface.

The instrument tip may comprise a shield mounted around the static portion. The shield may comprise an insulting covering mounted around the static portion. For example, the insulating shield may cover the support arm of the static portion. The insulating shield may also be using to partly cover the first electrode, e.g. to ensure that an exposed portion of the first electrode has a desired shape for controlling the delivery of RF or microwave energy. The insulating covering may have one or more field-shielding conductive regions, e.g. patches of metallisation on its outer surface. These conductive regions may provide shielding for the electric fields, e.g. to prevent leakage of energy from the instrument in unwanted locations. The shield may moulded over the instrument tip following assembly. Alternatively, the shield may be formed from a tube of insulating material that can be cut (e.g. laser cut) to the desired shape and then mounted over the blade elements. The shield may be formed from a suitable insulating plastic, e.g. PEEK or the like. The material for the shield may preferably be resistant to high temperatures.

The first blade element may be shaped as a longitudinally extending finger having an upstanding tooth at its distalmost end. The second blade element may be shaped in a corresponding way, e.g. as an elongate finger having a downwardly extending tooth at its distalmost end. The distalmost teeth may assist in retaining tissue in the gap between the jaws as they are closed. Additionally, the second blade element may be shaped to include a second downwardly extending tooth at a point in-between the distalmost end and proximalmost end. For example, the second downwardly extending tooth may be located at or near a midway point along the second blade element between the distalmost and proximalmost ends. The upstanding tooth and the two downwardly extending teeth may combine together to provide improved tissue retention in the gap between the jaws as they are closed.

A longitudinally extending insert may be mounted in the lumen of the flexible shaft to prevent relative movement of the actuator or coaxial cable with the shaft from resulting in lost or jerky movement of the instrument tip. The insert may comprise a tubular body having a plurality of longitudinal sub-lumens formed therein, wherein each of the plurality of longitudinal sub-lumens breaks the outer surface of the tubular body. The tubular body is sized to fit snugly within the lumen so that its broken circumferential surface defines a plurality of feet that abut the inner surface of the shaft to resist relative movement therebetween.

The coaxial transmission line may comprise a coaxial cable mounted in a first sub-lumen of the tubular body. The actuator may comprise a control rod slidably mounted in a second sub-lumen of the tubular body. The control rod may have a low friction coating (e.g. of PTFE or the like) to facilitate longitudinal sliding relative to the insert. Alternatively, the second sub-lumen may have a low friction tube (aka first tube) mounted therein, wherein the control rod can be slidably mounted in the low friction tube.

The electrosurgical resector tool may include a set of overlapping tubes which together provide a channel through which the control rod may slide to open and close the jaws. The set of overlapping tubes may be bonded to the instrument tip (e.g. the static portion) such that the control rod can slide within the channel in a predictable and reliable manner. For example, movement of the channel relative to the instrument tip is prevented which could otherwise interfere with the smooth movement (e.g. sliding) of the control rod and, by association, the smooth opening and closing of the jaws. Specifically, there may be provided a first tube (aka guide wire tube), a second tube (aka distal guide wire tube) and a third tube (aka short base tube). The first tube surrounds a majority of the control rod except a distal end region of the control rod. The first tube may surround a majority or entirety of the control rod except the distal end region. That is, the first tube may extend proximally all the way to, and possibly inside of, a handpiece for manually controlling opening and closing of the jaws. The distal end region of the control rod may be the final 4 mm to 8 mm (e.g. 5 mm). The first tube may be formed from PTFE or the like. The second tube surrounds the distal end region of the control rod except the attachment feature of the control rod, and the second tube protrudes proximally into the first tube to define an overlap region where the first tube overlaps the second tube. The attachment feature may account for the distalmost 2 mm or less of the control rod. A length of the overlap region may be about half of the length of the second tube, for example, the overlap region may be about 4 mm to 6 mm long, and the length of the second tube may be about 8 mm to 12 mm. The second tube may be formed from PTFE or the like. Also, the third tube surrounds the overlap region and a proximal end region of the static portion. A length of the overlap region may be about half of the length of the third tube, for example, the overlap region may be about 4 mm to 6 mm long, and the length of the third tube may be about 8 mm to 12 mm. The third tube may be formed from polyether block amide (aka PEBA, PEBAX or thermoplastic elastomer). The first, second and third tubes may be bonded to each other and to the static portion. Bonding may be via glue or adhesive, and/or via an interference fit between the overlapping tubes. For instance, the first, second and third tubes may be substantially clear (i.e. transparent) and may be bonded to the instrument tip (e.g. the static portion) by ultra-violet adhesive.

The instrument tip may be dimensioned to fit within an instrument channel of a surgical scoping device. Accordingly, a second aspect the invention provides an electrosurgical apparatus comprising: an electrosurgical generator for supplying EM energy; a surgical scoping device having an instrument cord for insertion into a patient's body, the instrument cord having an instrument channel extending therethrough; and an electrosurgical resector tool of the first aspect inserted through the instrument channel of the surgical scoping device.

Optionally, the electrosurgical generator is capable of supplying radiofrequency (RF) EM energy and microwave EM energy.

According to a third aspect of the invention, there is provided an electrosurgical resector tool comprising: a shaft defining a lumen; an energy conveying structure for carrying electromagnetic (EM) energy through the lumen of the shaft; an instrument tip mounted at a distal end of the shaft, wherein the instrument tip comprises: a static portion comprising a first blade element; and a movable portion comprising a second blade element, wherein the movable portion is movable relative to the static portion between a closed position in which the first blade element and second blade element lie alongside each other to an open position in which the second blade element is spaced from the first blade element by a gap for receiving biological tissue; a first electrode, a second electrode and a planar dielectric body, the first and second electrodes being spaced apart and electrically isolated from each other by the planar dielectric body, and wherein the first electrode and the second electrode are connected to the energy conveying structure for delivery of the EM energy from the instrument tip; an actuator for controlling relative movement between the movable portion and the static portion, the actuator comprising a control rod slidably mounted in the shaft, the control rod having an attachment feature engaged with the movable portion, whereby longitudinal movement of the control rod in the shaft causes movement of the movable portion relative to the static portion; and a first tube, a second tube and a third tube, wherein the first tube surrounds the control rod except a distal end region of the control rod, wherein the second tube surrounds the distal end region of the control rod except the attachment feature of the control rod, and the second tube protrudes proximally into the first tube to define an overlap region where the first tube overlaps the second tube, and wherein the third tube surrounds the overlap region and a proximal end region of the static portion.

The third aspect is analogous to the first aspect other than that: (i) the travel limiting mechanism is optional in the third aspect; and, (ii) the first, second and third tubes are essential in the third aspect. The further features and advantages of the first aspect are equally applicable and are hereby restated in respect of the second aspect.

The term “surgical scoping device” may be used herein to mean any surgical device provided with an insertion tube that is a rigid or flexible (e.g. steerable) conduit that is introduced into a patient's body during an invasive procedure. The insertion tube may include the instrument channel and an optical channel (e.g. for transmitting light to illuminate and/or capture images of a treatment site at the distal end of the insertion tube. The instrument channel may have a diameter suitable for receiving invasive surgical tools. The diameter of the instrument channel may be 5 mm or less.

Herein, the term “inner” means radially closer to the centre (e.g. axis) of the instrument channel and/or coaxial cable. The term “outer” means radially further from the centre (axis) of the instrument channel and/or coaxial cable.

The term “conductive” is used herein to mean electrically conductive, unless the context dictates otherwise.

Herein, the terms “proximal” and “distal” refer to the ends of the elongate probe. In use the proximal end is closer to a generator for providing the RF and/or microwave energy, whereas the distal end is further from the generator.

In this specification “microwave” may be used broadly to indicate a frequency range of 400 MHz to 100 GHz, but preferably the range 1 GHz to 60 GHz. Specific frequencies that have been considered are: 915 MHz, 2.45 GHz, 3.3 GHz, 5.8 GHz, 10 GHz, 14.5 GHz and 24 GHz. In contrast, this specification uses “radiofrequency” or “RF” to indicate a frequency range that is at least three orders of magnitude lower, e.g. up to 300 MHz, preferably 10 kHz to 1 MHz, and most preferably 400 kHz.

### BRIEF DESCRIPTION OF THE DRAWINGS

Embodiments of the invention are discussed in detail with reference to the accompanying drawings, in which:

FIG. 1 is a schematic diagram of an electrosurgical system that is an embodiment of the invention;

FIG. 2A is a perspective view of an instrument tip of an electrosurgical resector instrument that is an embodiment the invention in a closed configuration;

FIG. 2B is a side view of the instrument tip of FIG. 2A in the closed configuration;

FIG. 2C is a side view of the instrument tip of FIG. 2A in an open configuration;

FIG. 2D is a perspective view of the instrument tip of FIG. 2A in the open configuration;

FIGS. 3A and 3B are side and perspective views, respectively, of the instrument tip of FIG. 2A but with an outer sleeve removed to reveal internal parts;

FIG. 4 is a schematic partially cut-away side view of an electrosurgical resector instrument that is an embodiment the invention;

FIG. 5 is a reproduction of FIG. 2D but including labels corresponding to FIG. 4, to illustrate how the schematic view of FIG. 4 can translate onto the instrument tip of FIG. 2A;

FIG. 6A is a perspective view of the contents of an instrument shaft that can be used with an electrosurgical resector instrument that is an embodiment of the invention; and

FIG. 6B is a cross-section of the instrument shaft shown in FIG. 6A.

### DETAILED DESCRIPTION OF THE DRAWINGS

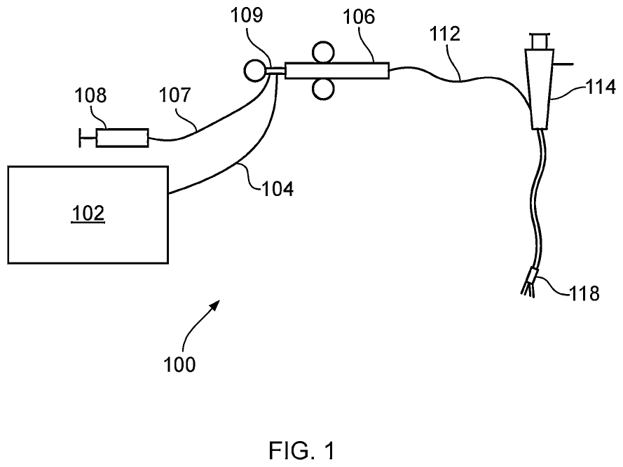

FIG. 1 is a schematic diagram of a complete electrosurgical system 100 that is an embodiment of the invention. The system is arranged to treat (e.g. cut or seal) biological tissue using electromagnetic (EM) energy (e.g. radiofrequency (RF) and/or microwave EM energy) from an instrument tip. The system 100 comprises a generator 102 for controllably supplying the EM energy (e.g. RF and/or microwave EM energy). A suitable generator for this purpose is described in WO 2012/076844, which is incorporated herein by reference. The generator 102 is connected to a handpiece 106 by an interface cable 104. The handpiece 106 may also be connected to receive a fluid supply 107 from a fluid delivery device 108, such as a syringe, although this is not essential. If needed, the handpiece 106 may house an instrument actuation mechanism that is operable by an actuator 109, e.g. a thumb operated slider or plunger. For example the instrument actuation mechanism may be used to operate a pivotable blade element of a resector instrument as discussed herein. Other mechanisms may also be included in the handpiece. For example, a needle movement mechanism may be provided (operable by a suitable trigger on the handpiece) for deploying a needle at the instrument. A function of the handpiece 106 is to combine the inputs from the generator 102, fluid delivery device 108 and instrument actuation mechanism, together with any other inputs which may be required, into a single flexible shaft 112, which extends from the distal end of the handpiece 106. The handpiece 106 may be as described in GB2567480.

The flexible shaft 112 is insertable through the entire length of an instrument (working) channel of a surgical scoping device 114. The flexible shaft 112 has an instrument tip 118 that is shaped to pass through the instrument channel of the surgical scoping device 114 and protrude (e.g. inside the patient) at the distal end of the endoscope's insertion tube. The instrument tip 118 includes a pair of blade elements for gripping biological tissue and an energy delivery structure arranged to deliver EM energy (e.g. RF and/or microwave EM energy) conveyed from the generator 102. Optionally the instrument tip 118 may also include a retractable hypodermic needle for delivering fluid conveyed from the fluid delivery device 108. The handpiece 106 includes an actuation mechanism for opening and closing the blade elements of the instrument tip 118. The handpiece 106 may also include a rotation mechanism for rotating the instrument tip 118 relative to the instrument channel of the surgical scoping device 114.

The structure of the instrument tip 118 may be arranged to have a maximum outer diameter suitable for passing through the working channel. Typically, the diameter of a working channel in a surgical scoping device such as an endoscope is less than 4.0 mm, e.g. any one of 2.8 mm, 3.2 mm, 3.7 mm, 3.8 mm. The flexible shaft 112 may have a maximum diameter less than this, e.g. 2.65 mm. The length of the flexible shaft 112 can be equal to or greater than 1.2 m, e.g. 2 m or more. In other examples, the instrument tip 118 may be mounted at the distal end of the flexible shaft 112 after the shaft has been inserted through the working channel (and before the instrument cord is introduced into the patient). Alternatively, the flexible shaft 112 can be inserted into the working channel from the distal end before making its proximal connections. In these arrangements, the distal end assembly 118 can be permitted to have dimensions greater than the working channel of the surgical scoping device 114. The system described above is one way of introducing the instrument into a patient. Other techniques are possible. For example, the instrument may also be inserted using a catheter.

Although the examples herein are present in the context of a surgical scoping device, it is to be understood that the electrosurgical resector instrument may be embodiment in a device suitable for open surgery or use with a laparoscope.

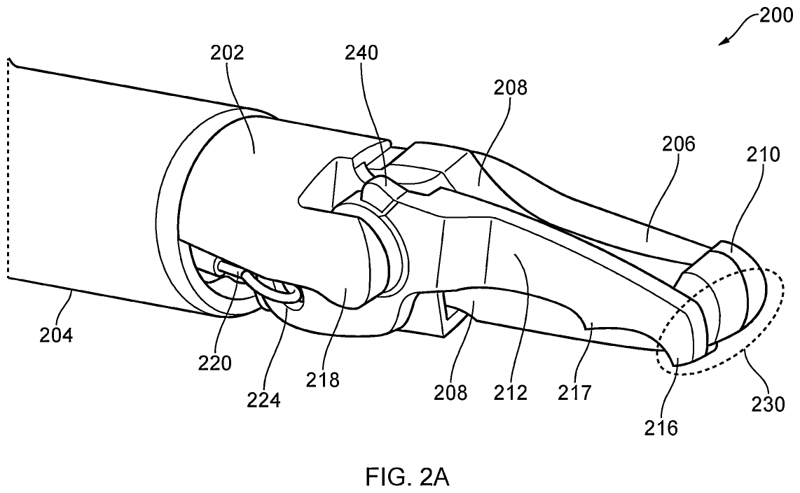

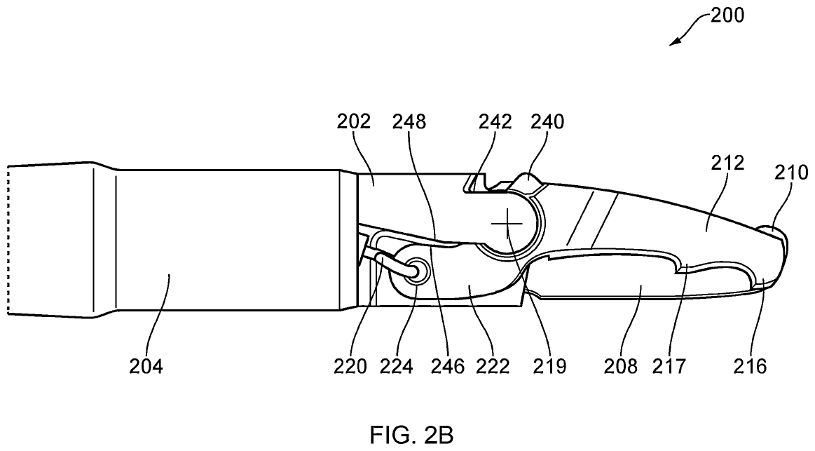

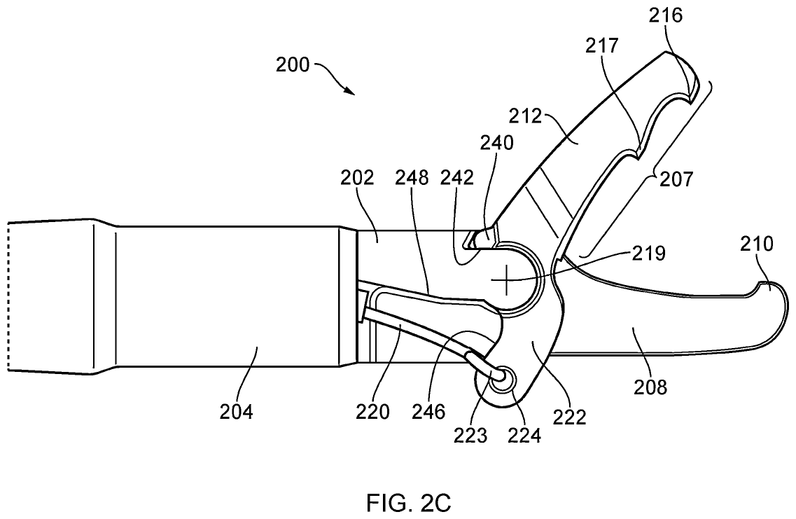

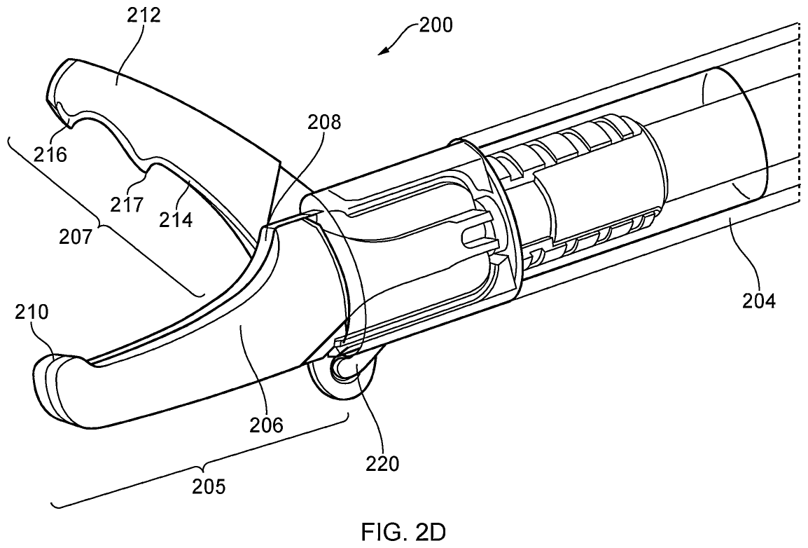

FIGS. 2A-2D are different views of an instrument tip 200 of an electrosurgical resector instrument that is an embodiment the invention. FIG. 2A is an isometric view of the instrument tip 200 in a closed position, FIG. 2B is a side view of the instrument tip 200 in the closed position, FIG. 2C is a side view of the instrument tip 200 in an open position, and FIG. 2D is another side view of the instrument tip 200 in the open position. The instrument tip 200 is mounted at the distal end of a flexible shaft 204, which may correspond to the flexible shaft 112 discussed above. In this embodiment, the instrument tip 200 comprises a static portion 202 that carries a first electrode 206 (e.g. see FIG. 2D), and a movable portion 212 that carries a second electrode 214 (e.g. see FIG. 2D). However, the invention need not be limited to this configuration. In other examples both electrodes may be provided on either the static portion 202 or the movable portion 212.

The static portion 202 has a proximal region that is secured to a distal end of the flexible shaft 204. The static portion 202 extends in a longitudinal direction away from the distal end of the flexible shaft 204. At its distal end, the static portion 202 defines a first blade element 205, which is a longitudinally extending finger having an upstanding tooth 210 at its distalmost end. The first electrode 206 extends along a lateral surface of the first blade element 205. However, in another embodiment, the first electrode 206 could instead extend along only an upper surface of the first blade element 205.

The movable portion 212 is pivotably mounted on the static portion 202. In this embodiment, the movable portion 212 comprises a second blade element 207 (e.g. see FIG. 2D), which is an elongate finger having a length commensurate with the first blade element 205. The second blade element 207 has a first downwardly extending tooth 216 at its distalmost end. Additionally, the second blade element 207 has a second downwardly extending tooth 217 approximately midway along the second blade element 207.

The movable portion is pivotable about a pivot axis 219 (see FIGS. 2B and 2C) located at a proximal end of the first blade element 205, whereby the second blade element 207 can swing between an open position (shown FIGS. 2C and 2D) in which it is angled away from the first blade element 205 and a closed position (shown in FIGS. 2A and 2B) where is lies alongside (i.e. laterally adjacent) to the first blade element 205. The range of movement of the movable portion may be such to allow the second blade element 207 to adopt an acute angle relative to the first blade element 205, e.g. about 60 degrees. This may be particular useful for ensuring that the jaws do not over extend so that the opening and closing mechanism remains smooth and consistent throughout the entire range of motion of the jaws.

The first blade element 205 and second blade element 207 may thus define a scissor-type closure mechanism in which tissue located in a gap between the blade elements 205, 207 when in the open position can have pressure applied to it as the second blade element 207 is moved to the closed position. The upstanding tooth 210 on the first blade element 205 and the downwardly extending teeth 216, 217 on the second blade element 207 act to retain tissue in the gap as second blade element 207 moves to the closed position.

The first blade element 205 comprises a planar dielectric body 208, e.g. made from ceramic or other suitable electrically insulating material. The planar dielectric body 208 defines a plane that is parallel to a plane through which the second blade element 207 pivots. The planar dielectric body 208 provides an insulating barrier between the first electrode 206 and the second blade element 207. For example, the second blade element 207 is arranged to slide past a first surface of the planar dielectric body 208, and the first electrode 206 is formed on a second surface of the planar dielectric body 208, the second surface being on the opposite side of the planar dielectric body 208 from the first surface. The first electrode 206 may be made from a conductor which exhibits high conductivity, e.g. gold or the like.

The second electrode 214 extends along a side surface of the second blade element 207 that slides past an adjacent side surface of the first blade element 205 (i.e. the first surface of the planar dielectric body 208 mentioned above) when the second blade element 207 is moved into the closed position. The second electrode 214 extends along the inside laterally facing surface of the movable portion 212. The second blade element 207, and the movable portion 212, may be formed from an electrically conductive material that is coated with an insulating material. For example, it may be made from stainless steel with a ceramic (e.g. alumina), diamond-like carbon (DLC) coating, enamel coating, or a silicon-based paint coating. Next, the material may be further coated with Parylene N in order to seal the insulating coating. For example, the Parylene N coating may have a thickness of between 2 and 10 micrometers, and preferably between about 3 and 7 micrometers, and more preferably about 5 micrometers. The Parylene N coating penetrates the pores in the insulator coating and effectively makes it waterproof. In turn, this increases the breakdown voltage of the insulator coating when it is wet. The insulating coating and Parylene N coating may be removed, e.g. etched away, from regions where it is not required. For example, the second electrode 214 may be formed by etching away the insulating coating and Parylene N coating from the inside bottom edge of the movable portion 212. A gold layer may be deposited over the etched surface to form the electrode. Other portions of the coatings may be removed to enable an electrical connection to be made to the outer conductor of the coaxial cable, as explained below.

The flexible shaft 204 defines a lumen through which extends a coaxial cable (not shown) for conveying EM energy (e.g. RF and/or microwave EM energy), and a longitudinally slidable control rod (shown in FIG. 2C) for controlling movement of the movable portion 212.

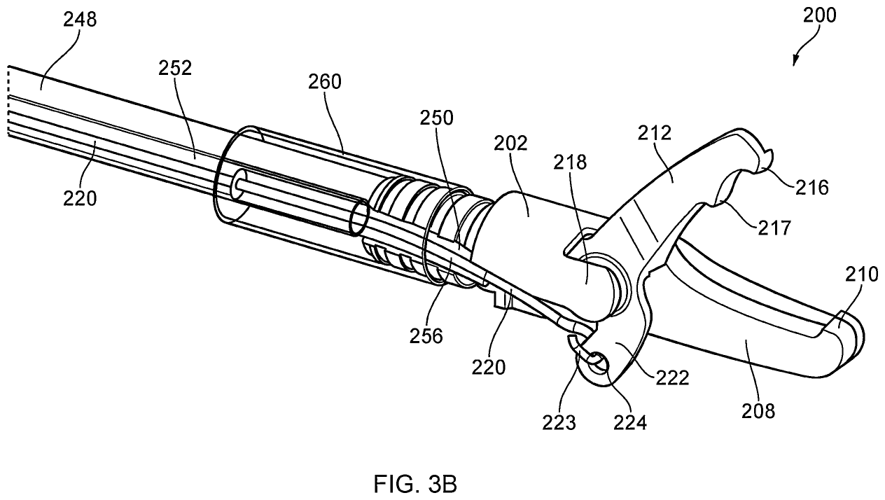

As discussed in more detail with reference to FIG. 4, the first electrode 206 is electrically connected to an inner conductor of a coaxial cable inside the shaft 204 and the second electrode 214 is electrically connected to an outer conductor of the coaxial cable. The instrument tip thus provides an energy delivery structure that is operable to deliver EM energy. For example, RF energy may be delivered along a current path (e.g. through tissue) between the first electrode and second electrode, and/or microwave energy may be delivered through a microwave field emitted by the first electrode and second electrode.

The instrument tip 200 may provide three operational modalities. In a first modality, the instrument can be used with the blade elements 205, 207 in the closed position to deliver RF EM energy to cut through biological tissue. In this first modality, the RF EM energy passes primarily between the first electrode 206 and second electrode 214 in a distal cutting zone 230 adjacent to the upstanding tooth 210 on the first blade element 205 and the downwardly extending tooth 216 on the second blade element 207 (e.g. see FIG. 2A). The instrument may thus be used to sweep or glide across or through tissue to effect cutting.

In a second modality, the blade elements 205, 207 may be used to perform a grasping cut, i.e. a cut through tissue captured between the blade elements. In this modality cutting is done by a combination of physical pressure applied by closing the blade elements 205, 207 and RF EM energy applied during the closing process.

In a third modality, the blade elements 205, 207 may be used to grasp and seal tissue, such as a blood vessel or the like. In this modality, microwave EM energy is delivered to the electrodes, which set up a microwave field that acts to coagulate the tissue held within the blade elements.

The static portion 202 may have a dielectric shield mounted over its outer surface. In this example, the dielectric shield is a thermoplastic polymer, e.g. polyether ether ketone (PEEK), or the like. The dielectric shield may be moulded over the device, or may be a cover (e.g. formed by laser cutting a suitably size tube) that can slide over the instrument tip when the blade elements are in the closed position. The dielectric shield can be used to control the shape of the first electrode 206, e.g. to ensure that the first electrode 206 is exposed substantially only at an upper surface of the first blade element 205. In turn this can ensure that the EM energy (e.g. RF and/or microwave energy) delivered from the electrodes is focussed into the desired region.

The opening and closing operation of the instrument tip 200 will now be described with reference to FIGS. 2A to 2D.

FIGS. 2C and 2D illustrate the instrument tip 200 in an open position, with the movable portion 212 disposed so that the second blade element 207 is sits at an acute angle (e.g. 60 degrees) to the first blade element 205. As seen best in FIG. 2A, the static portion 202 includes a longitudinally extending arm 218 that provides a pivot base to which the movable portion 212 is attached. The arm 218 has a pivot axle (not shown) rotatably mounted therein. The pivot axle defines the laterally extending pivot axis 219 (i.e. the pivot axis is orthogonal to the longitudinal direction defined by the flexible shaft 204).

The support arm 218 is formed on the static portion 202 so as to define a slot in the static portion 202. The slot may be necessary in order to provide space for part of the moveable portion 212 (e.g. a proximal part, such as attachment plate 222) to move relative to the static portion 202 as the movable portion 212 moves between the open and closed positions. The static portion 202 and the support arm 218 may form part of an electrical connection between a conductor in the shaft 204 and the second electrode 214. For example, the static portion 202 (e.g. the support arm 218) may be formed from an insulator-coated conductive material which is further coated with parylene N, and may comprise a proximal contact portion at which the insulator coating and the parylene N coating is removed and which is electrically connected to the conductor in the shaft 204. For example, the Parylene N coating may have a thickness of between 2 and 10 micrometers, and preferably between about 3 and 7 micrometers, and more preferably about 5 micrometers. As mentioned above, the Parylene N coating may be used to improve waterproofness and increase breakdown voltage of the insulating coating in wet conditions. In order to facilitate the creation of coatings which cover the required areas of the static portion 202 and are uniform, it may be beneficial to limit certain dimensions of the slot so that the coating materials can penetrate all interior surfaces of the slot. Thus, a length of the slot (i.e. the dimension in line with the length of shaft 204) may be between lmm and 3 mm (preferably less than 2 mm). A width of the slot (i.e. the dimension in line with the pivot axis 219) may be between 0.2 mm and 1.2 mm (preferably more than 0.7 mm). A depth of the slot may be between 0.2 mm and 1.2 mm (preferably more than 0.6 mm).

The slidable control rod 220 protrudes from the flexible shaft 204. The static portion 202 has a guide channel (not shown) formed therein through which the control rod 220 passes. The control rod 220 has a distal attachment feature 223 that is engaged with the movable portion 212. In this example, the distal attachment feature 223 is a hook that engages a circular aperture 224 formed in an attachment plate 222 of the movable portion 212. Other types of engagement may be used. Longitudinal sliding motion of the control rod 220 is transformed into pivoting motion of the attachment plate 222. The attachment plate 222 may be integrally formed with or otherwise operably coupled to the second blade element 207. The attachment feature 223 and aperture 224 may be formed such that longitudinal movement of the attachment feature 223 in the aperture 224 is substantially prevented. For example, the control rod diameter may be only slightly less than a diameter of the aperture 224 such that the attachment feature 223 can rotate within the aperture 224 but cannot move longitudinally within the aperture 224. In this way, all longitudinal movement of the control rod can be translated into movement of the jaws.

FIGS. 2A and 2B show the instrument tip 200 in the closed position. Moving from the open position of FIGS. 2C and 2D to the closed position of FIGS. 2A and 2B is achieved by retracting the control rod 220 into the flexible sleeve 204, for example, via a handpiece such as handpiece 106 of FIG. 1.

Also shown in FIGS. 2A to 2D is a travel limiting mechanism of the instrument tip 200. The travel limiting mechanism operates to limit a maximum extent of relative movement between the second blade element 207 and the first blade element 205 in the open position and the closed position.

As seen best in FIGS. 2B and 2C, the static portion 202 and the movable portion 212 together include at least one pair of cooperating structures arranged to provide the travel limiting mechanism. A first pair of cooperating structures includes a raised protrusion (or shoulder) 240 and a cooperating stop surface 242. The raised protrusion 240 is formed on a top surface of the moveable portion 212 and distally of a connection between the movable portion and the static portion (e.g. distally of the pivot axis 219). Also, the stop surface 242 is formed on a top surface of the static portion 202 and proximally of the connection between the movable portion and the static portion. In an embodiment, the stop surface 242 is formed on the top surface of the support arm 218 of the static portion 202.

As seen on FIG. 2C, in use, the raised protrusion 240 and the stop surface 242 are configured to abut each other in the open position. In this way, the first pair of cooperating structures limits a maximum extend of relative movement between the second blade element 207 and the first blade element 205 in the open position. That is, the first pair of cooperating structures limits how wide the jaws may open. In an embodiment, the first pair of cooperating structure are configured (e.g. sized, shaped, positioned) to limit a maximum angle between the first and second blade elements to be about 60 degrees. It is noted that in the absence of the first pair of cooperating structures, the jaw may be able to open wider. Therefore, the first pair of cooperating structures may limit how wide the jaws can open in order to ensure that jaw operation (e.g. movement) is consistent and reliable throughout the entire permitted range of travel. For example, in the absence of the first pair of cooperating structures, the extremes of the range of movement of second blade element may become jerky and may exert proportionally more strain on the instrument tip compared to the middle portion of the range of movement. Thus, by limiting the maximum extend by which the second blade element can rotate away from the first blade element, the overall movement of the jaws can be kept more consistent and reliable. Additionally, it may be desirable to limit how far the jaws can open such that they do not get stuck or locked in the open position. Further, it may be desirable to limit how far the jaws can open such that the overall profile of the instrument tip can be kept smaller which may be beneficial in tight spaces or locations. Such advantages are particularly important in precision surgical operations.

In the embodiment shown, the moveable portion comprises the raised protrusion and the static portion comprises the stop surface. However, it is to be understood that in at least some other embodiments, the raised protrusion may be located on the static portion and the stop surface may be located on the moveable portion. Additionally, in some other embodiments, the first pair of cooperating structures may include two raised protrusions, rather than a raised protrusion and a stop surface.

Additionally, the travel limiting mechanism may include a second pair of cooperating structures that includes a pair of abutment surfaces 246 and 248. The abutment surface 246 is formed on a top surface of the movable portion 212 and proximally of a connection between the moveable portion 212 and the static portion 202 (e.g. proximally of the pivot axis 219). The abutment surface 248 is formed on an under surface of the static portion 202 and proximally of the connection between the moveable portion 212 and the static portion 202. In an embodiment, the abutment surface 248 is formed on an underside of the support arm 218.

As seen in FIG. 2B, in use, the second pair of cooperating structures are configured to abut each other in parallel formation in the closed position. That is, in the closed position, the abutment surface 246 is substantially parallel to and in contact with the abutment surface 248. In this way the moveable portion 212 and the second blade element 207 are prevented from moving further past the static portion 202 and the first blade element 205. As such, the second pair of cooperating structures limit the relative positions of the moveable portion 212 and the second blade element 207 with respect to the static portion 202 and the first blade element 205 in the closed position. For example, the second pair of cooperating structures may be configured (e.g. sized, shaped, positioned) to ensure that the second blade element 207 (e.g. tooth 216 and/or tooth 217) does not protrude below the planar dielectric body 208 in the closed position. For instance, a dimension (e.g. length or width) of the attachment plate 222 may be set to define the closed position. It is noted that in the absence of the second pair of cooperating structures, the second blade element 207 may be able to protrude below the bottom surface of the planar dielectric body 208 (e.g. considering the orientation shown in FIG. 2B). This may be undesirable since it may cause unintended damage to tissue located at the underside of the instrument tip 200. Also, if the second blade element 207 is able to pivot past and below the planar dielectric body 208, subsequent opening of the jaws may unintentionally cut any tissue which is positioned in-between the top surface of the distal tip of the movable portion 212 and the bottom surface of the distal tip of the static portion 202.

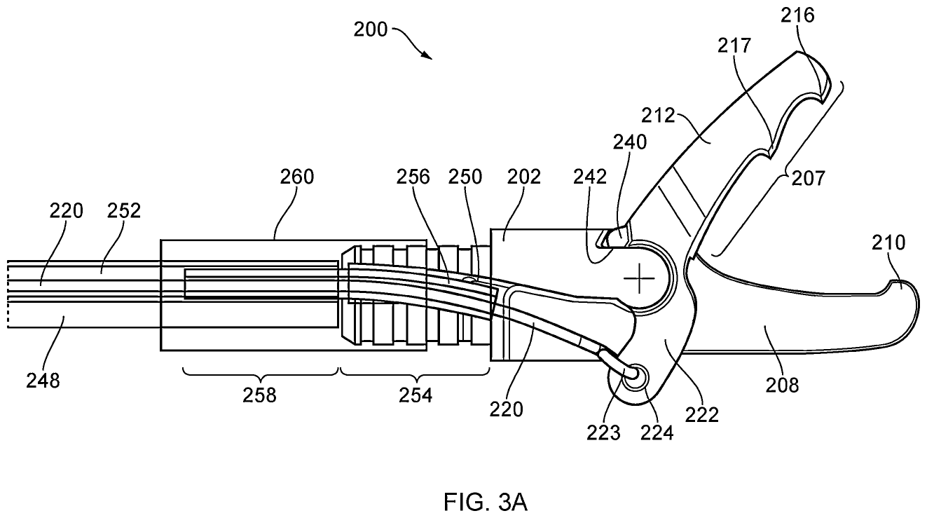

FIGS. 3A and 3B illustrate a mechanism for coupling the control rod 220 to the instrument tip 200. In FIGS. 3A and 3B an outer sleeve of the shaft 204 has been omitted for clarity so that the elements beneath are visible. It is to be understood that after the arrangement of FIGS. 3A and 3B has been formed, an outer sleeve would be added, as shown in FIGS. 2A to 2C.

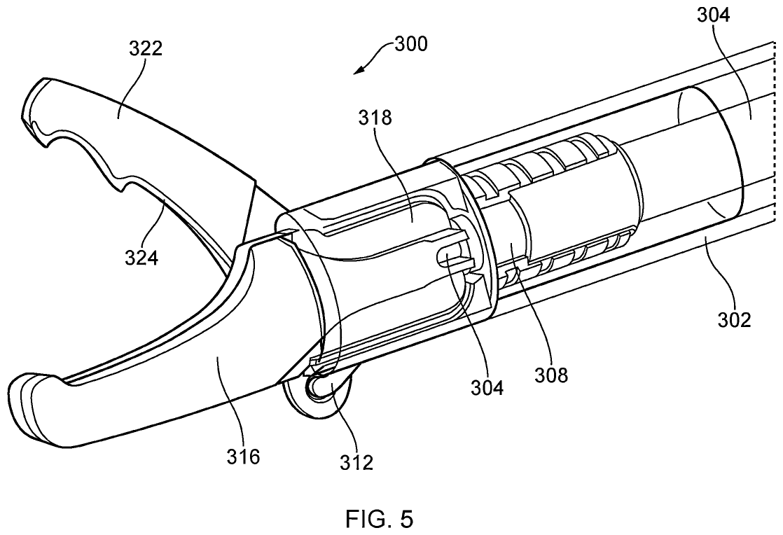

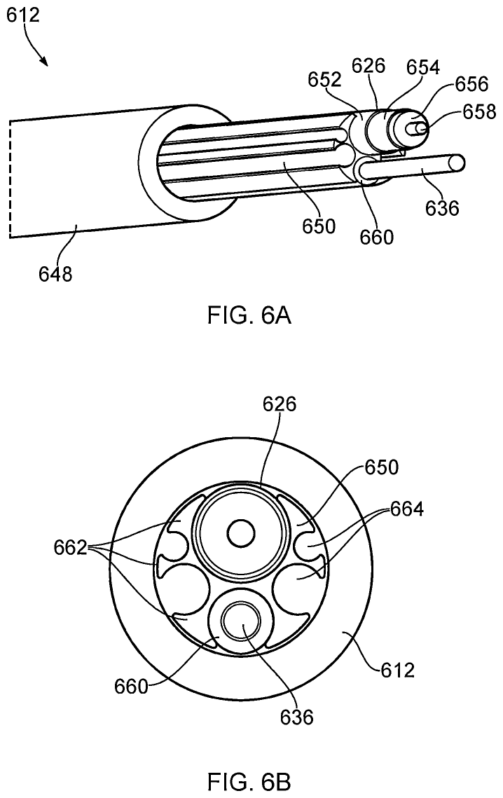

In FIGS. 3A and 3B, the elements of the instrument tip 200 are as described above, and corresponding reference signs are shown. FIGS. 3A and 3B illustrate how the control rod 220 extends from its connection with the movable portion 212 into the shaft. Furthermore, the static portion 202 includes a guide channel 250 which receives the control rod 220. At least a portion of the guide channel 250 may be substantially U-shaped to accommodate the control rod 220. The coaxial cable 248 can be seen behind the control rod 220 along the length of the shaft. As is explained below with reference to FIGS. 6A and 6B, the control rod 220 extends along the shaft 204 within a guide wire tube (aka first tube) 252. The guide wire tube 252 ensures that the control rod 220 moves smoothly (i.e. with reduced friction) within the shaft 204. The proximal end of the guide wire tube 252 terminates at or inside a handpiece (e.g. handpiece 106 of FIG. 1). The distal end of the guide wire tube 252 terminates at (i.e. just before) the proximal end of the static portion 202, as shown in FIGS. 3A and 3B. A proximal end region 254 of the static portion 202 may have a generally circular cross-section and have a reduced width (e.g. diameter) compared to features of the static portion 202 which are positioned distally of it. Also, the proximal end region 254 may include one or more surface ribs. Since a distal end of the guide wire tube 252 terminates just before the proximal end of the static portion 202, the guide wire tube 252 surrounds a majority or an entirety of the control rod 220 except a distal end region of the control rod 220. The distal end region of the control rod 220 may be the final 4 mm to 8 mm.

A distal guide wire tube (aka second tube) 256 surrounds the distal end region of the control rod 220 except the attachment feature 223 of the control rod 220. The attachment feature may account for the distalmost 2 mm or less of the control rod 220. Also, the distal guide wire tube 256 protrudes proximally into the guide wire tube 252 to define an overlap region 258 where the guide wire tube 252 overlaps the distal guide wire tube 256. A length of the overlap region 258 may be about half of the length of the distal guide wire tube 256, for example, the overlap region 250 may be about 4 mm to 6 mm long, and the length of the distal guide wire tube 256 may be about 8 mm to 12 mm.

A base short tube (aka third tube) 260 surrounds the overlap region 258 and a proximal part of proximal end region 254 of the static portion 202. The base short tube 260 fits around the proximal end region 254 and may be held in place by frictional engagement which is enhanced by the aforementioned ribs. A length of the overlap region 258 may be about half of the length of the base short tube 260, and a proximal end of the base short tube 260 may extend proximally past the proximal end of the overlap region 258. For example, the overlap region 258 may be about 4 mm to 6 mm long, and the length of the base short tube 260 may be about 8 mm to 12 mm. The base short tube 260 is then bonded to the proximal end region 254 and to both the guide wire tube 252 and the distal guide wire tube 256. For example, bonding may be via an interference fit and/or an adhesive. In an embodiment, the three tubes are transparent and they are bonded together and to the proximal end region using an ultra-violet adhesive. The aforementioned rib features on the proximal end region 254 may help to ensure that the base short tube 260 remains attached to the static portion 202.

Accordingly, the control rod 220 free to slide within a channel formed by the guide wire tube 252 and the distal guide wire tube 256. As such the control rod 220 does not snag or catch on any features as it is deployed and retracted within the shaft 204 to open and close the jaws. Also, this channel extends through the connection between the shaft 204 and the static portion 202 meaning that snagging and catching is also prevented as the control rod 220 moves relative to the static portion 202. Further, the base short tube 260 fixes the channel relative to the instrument tip 200 meaning that the channel cannot move relative to the instrument tip 200. In turn, this ensures that the movement of the control rod 220 remains smooth and consistent.

It is noted that as a final step, an outer sleeve of the shaft 204 is positioned over the top of the base short tube 206, as is shown in FIGS. 2A to 2C. The outer sleeve may be bonded in position by adhesive. In an embodiment, the guide wire tube 252 and the distal guide wire tube 256 may be formed from PTFE or the like. On the other hand, the base short tube may be formed from polyether block amide (aka PEBA, PEBAX or thermoplastic elastomer).

FIG. 4 is a schematic partly cut-away side view of an instrument tip 300 for an electrosurgical resector instrument that is an embodiment of the invention. The instrument tip 300 is located at the distal end of a flexible sleeve 302, which conveys a coaxial cable 304 and a control rod 312. The control rod 312 is for controlling pivoting motion of a movable portion 322 relative to a static portion 318 in the same way as discussed above. The static portion 318 has a planar dielectric body 314 secured to it, e.g. by a suitable adhesive, the planar dielectric body 314 extending in a longitudinal direction away from the static portion 318 to form a first blade element. A first electrode 316 is formed on one side of the planar dielectric body 314.

The moveable portion 322 is pivotably mounted on the static portion 318 via a pivot axle (not visible in FIG. 4) at an opposite side of the planar dielectric body 314 to the first electrode 316. The moveable portion 322 comprises a second blade element that is arrange to slide past the first blade element in a similar manner to the first and second blade elements 205, 207 discussed above. The moveable portion 322 includes a second electrode 324 thereon that lies adjacent the opposite side of the planar dielectric body 314 when the blade elements are in a closed position.

The coaxial cable 304 comprises an inner conductor 306 that is separated from an outer conductor 310 by a dielectric material 308. The dielectric material 308 and inner conductor 306 extend beyond a distal end of the outer conductor 310. A distal end of the dielectric material 308 abuts a proximal end of the planar dielectric body 314. The inner conductor 306 extends distally from this junction to overlap with and electrically contact a proximal portion of the first electrode 316. The invention need not be limited to this arrangement. In other examples, the inner conductor may be electrically connected to an electrode on the movable portion, for example.

The static body 318 includes a support arm on which the movable portion is mounted. The planar dielectric body 314 may also be mounted on the support arm, e.g. using adhesive of the like. The static portion (e.g. the support arm) is formed from an electrically conductive material (e.g. stainless steel) with an electrically insulating coating. As mentioned above, this insulating coating may be further coated with Parylene N in order to improve waterproofness and increase breakdown voltage of the insulating coating in wet conditions. The coatings are removed at a proximal contact portion 320 which is electrically connected to the outer conductor 310 of the coaxial cable 304. The movable portion 322 is also formed from an electrically conductive material (e.g. stainless steel) with an electrically insulating coating. Again, this insulating coating may be further coated with Parylene N. The movable portion 322 is physically engaged with the static portion 318 at the pivot connection. An electrical connection between the second electrode 324 and the outer conductor 310 of the coaxial cable 304 passes through the pivot connection. For example, the pivot axle itself may be formed from an electrical conductive material (e.g. stainless steel). The insulating coating and the Parylene N coating of the static portion 318 may be removed at a region of sliding engagement (e.g. an aperture or recess for receiving the pivot axle) between the static portion 318 and the movable portion 322. Similarly, the insulating coating and the Parylene N coating of the movable portion 322 may be removed at this region. As the second electrode 324 may be or may be electrically connected to the electrically conductive material of the movable portion 322, a complete electrical connection to the outer conductor can be formed.

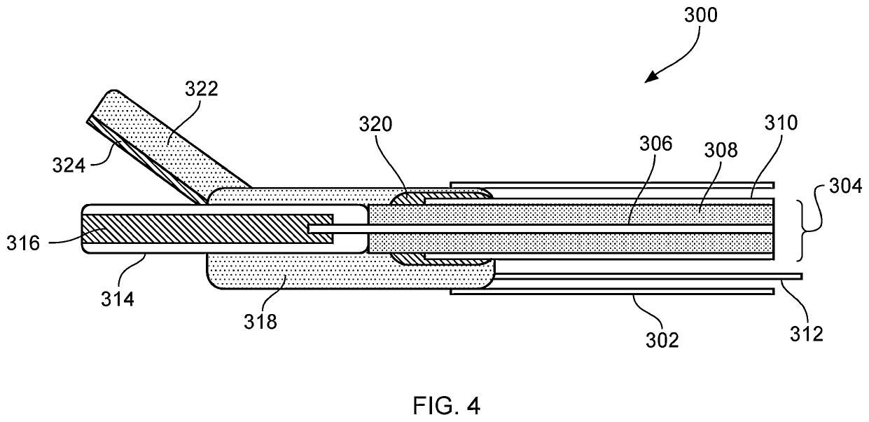

FIG. 5 is a reproduction of FIG. 2D and shows the shaft 302 as partly transparent to illustrate how the schematic features of FIG. 4 may map on to the device shown in FIGS. 2A 2D. Features in common with FIG. 4 are given the same reference numbers and are not described again.

FIG. 6A is a cut-away perspective view of the instrument shaft 612 as it travels towards the instrument tip. The instrument shaft 612 comprises an outer sleeve 648 that defines a lumen for conveying the coaxial cable 626 and control rod 636. In this example, the coaxial cable 626 and control rod 636 are retained in a longitudinally extending insert 650. The insert 650 is an extrusion, e.g. formed from a deformable polymer such as PEEK or other plastic with similar mechanical properties. As shown more clearly in FIG. 6B, the insert 650 is a cylindrical element having a series of sub-lumens 664 cut away around its outer surface. The sub-lumens 664 break through the outer surface of the insert 650 to define a plurality of discrete feet 662 around the circumference thereof. The sub-lumens 664 can be sized to convey components such as the coaxial cable 626 or control rod 636, or may be for the purpose of allowing fluid flow along the lumen of the sleeve 648.

It may be beneficial for the insert not to include any enclosed sub-lumens. Fully enclosed sub-lumens can be prone to retaining deformations if stored in a bent condition. Such deformations can lead to jerky motion in use.

The insert 650 may comprise a sub-lumen for receiving the coaxial cable 626. In this example, the coaxial cable 626 comprises an inner conductor 658 separated from an outer conductor 654 by a dielectric material 656. The outer conductor 654 may in turn have a protective cover or sheath 652, e.g. formed from PTFE or other suitably low friction material to permit relative longitudinal movement between the insert and coaxial cable as the shaft with flexing of the shaft.

Another sub-lumen may be arranged to receive a standard PFTE tube 660 through which the control rod 636 extends (this may be the guide wire tube 252 of FIGS. 3A and 3B). In an alternative embodiment, the control rod 636 may be provided with a low-friction (e.g. PFTE) coating before use, so that a separate PFTE tube is not required.

The insert is arranged to fill, i.e. fit snugly within, the lumen of the sleeve 648 when mounted with the coaxial cable 626 and control rod 636. This means that the insert functions to restrict relative movement between the coaxial cable, control rod and sleeve during bending and rotation of the shaft 612. Moreover, by filling the sleeve 648, the insert helps to prevent the sleeve from collapsing and losing rotation if rotated excessively. The insert is preferably made from a material that exhibits rigidity to resist such movement.

The presence of the insert may furthermore prevent “lost” travel of the control rod caused by deformation of the instrument shaft 612.

The extruded insert discussed above provides cam-like feet that jam on the inside of the sleeve and impede the wrapping of the control rod around the axis of the sleeve. This will reduce the lost travel discussed above.

1. An electrosurgical resector tool comprising:
a shaft defining a lumen;
an energy conveying structure for carrying electromagnetic (EM) energy through the lumen of the shaft;
an instrument tip mounted at a distal end of the shaft, wherein the instrument tip comprises:
a static portion comprising a first blade element; and
a movable portion comprising a second blade element, wherein the movable portion is movable relative to the static portion between a closed position in which the first blade element and second blade element lie alongside each other to an open position in which the second blade element is spaced from the first blade element by a gap for receiving biological tissue;
a travel limiting mechanism operable to limit a maximum extent of relative movement between the second blade element and the first blade element in the open position;
wherein the static portion and the movable portion together comprise at least one pair of cooperating structures arranged to provide the travel limiting mechanism;
wherein a first pair of cooperating structures comprises a raised protrusion and a cooperating stop surface, the raised protrusion and the stop surface being configured in use to abut each other in the open position;
a first electrode, a second electrode and a planar dielectric body, the first and second electrodes being spaced apart and electrically isolated from each other by the planar dielectric body, and wherein the first electrode and the second electrode are connected to the energy conveying structure for delivery of the EM energy from the instrument tip; and
an actuator for controlling relative movement between the movable portion and the static portion.

2. An electrosurgical resector tool according to claim 1, wherein the travel limiting mechanism is operable to limit the maximum extent of relative movement between the second blade element and the first blade element in the closed position;
wherein a second pair of cooperating structures includes a pair of abutment surfaces, the pair of abutment surfaces being configured in use to abut each other in parallel formation in the closed position.

3. An electrosurgical resector tool comprising:
a shaft defining a lumen;
an energy conveying structure for carrying electromagnetic (EM) energy through the lumen of the shaft;
an instrument tip mounted at a distal end of the shaft, wherein the instrument tip comprises:
a static portion comprising a first blade element; and
a movable portion comprising a second blade element, wherein the movable portion is movable relative to the static portion between a closed position in which the first blade element and second blade element lie alongside each other to an open position in which the second blade element is spaced from the first blade element by a gap for receiving biological tissue;
a travel limiting mechanism operable to limit a maximum extent of relative movement between the second blade element and the first blade element in the closed position;
wherein the static portion and the movable portion together comprise at least one pair of cooperating structures arranged to provide the travel limiting mechanism;
wherein a first pair of cooperating structures includes a pair of abutment surfaces, the pair of abutment surfaces being configured in use to abut each other in parallel formation in the closed position;
a first electrode, a second electrode and a planar dielectric body, the first and second electrodes being spaced apart and electrically isolated from each other by the planar dielectric body, and wherein the first electrode and the second electrode are connected to the energy conveying structure for delivery of the EM energy from the instrument tip; and
an actuator for controlling relative movement between the movable portion and the static portion.

4. An electrosurgical resector tool according to any one of claims 1 to 3, wherein one of the first blade element and the second blade element comprises the planar dielectric body extending longitudinally and having the first electrode on a first laterally facing surface thereof, and wherein, in the closed position, the other of the first blade element and the second blade element lies adjacent to a second laterally facing surface of the longitudinally extending planar dielectric body opposite to the first laterally facing surface thereof.

5. An electrosurgical resector tool according to claim 4, wherein the second electrode is located on the second laterally facing surface of the longitudinally extending planar dielectric body.

6. An electrosurgical resector tool according to claim 4, wherein the longitudinally extending planar dielectric body is on the first blade element, and wherein the second electrode extends along a side of the second blade element.

7. An electrosurgical resector tool according to claim 6, wherein the second blade element is formed from an insulator-coated conductive material which is further coated with parylene N, and wherein the second electrode is formed at a side portion of the second blade element where the insulator coating and the parylene N coating is removed.

8. An electrosurgical resector tool according to any preceding claim when dependent on claim 1 wherein the moveable portion comprises the raised protrusion and the static portion comprises the stop surface.

9. An electrosurgical resector tool according to claim 8, wherein the raised protrusion is formed on a top surface of the moveable portion and distally of a connection between the movable portion and the static portion, and wherein the stop surface is formed on a top surface of the static portion and proximally of the connection between the movable portion and the static portion.

10. An electrosurgical resector tool according to any one of claims 2 to 9, when dependent on claim 2 or 3, wherein a first abutment surface of the pair of abutment surfaces is formed on a top surface of the movable portion and proximally of a connection between the moveable portion and the static portion, and wherein a second abutment surface of the pair of abutment surfaces is formed on an under surface of the static portion and proximally of the connection between the moveable portion and the static portion.

11. An electrosurgical resector tool according to any preceding claim, wherein the static portion comprises a support arm on which the movable portion is mounted.

12. An electrosurgical resector tool according to claim 11, wherein the support arm defines a slot in the static portion for receiving part of the movable portion and wherein at least one of the following applies:
a length of the slot is less than 2 mm,
a width of the slot is more than 0.7 mm,
a depth of the slot is more than 0.6 mm.

13. An electrosurgical resector tool according to claim 11 or 12, wherein the static portion is formed from an insulator-coated conductive material which is further coated with parylene N, and wherein the support arm comprises a proximal contact portion at which the insulator coating and the parylene N coating is removed to form part of an electrical connection between the energy conveying structure and the second electrode.

14. An electrosurgical resector tool according to any preceding claim, when dependent on claim 1, wherein the movable portion is pivotable relative to the static portion, whereby the second blade element is angled relative to the first blade element in the open position, and wherein, in the open position, the travel limiting mechanism is arranged to limit a maximum angle between the first and second blade elements to 60 degrees.

15. An electrosurgical resector tool according to any preceding claim, wherein the actuator comprises a control rod slidably mounted in the shaft, the control rod having an attachment feature engaged with the movable portion, whereby longitudinal movement of the control rod in the shaft causes movement of the movable portion relative to the static portion.

16. An electrosurgical resector tool according to claim 15, further comprising a first tube, a second tube and a third tube, wherein the first tube surrounds the control rod except a distal end region of the control rod, wherein the second tube surrounds the distal end region of the control rod except the attachment feature of the control rod, and the second tube protrudes proximally into the first tube to define an overlap region where the first tube overlaps the second tube, and wherein the third tube surrounds the overlap region and a proximal end region of the static portion.

17. An electrosurgical resector tool according to claim 16, wherein the first, second and third tubes are substantially clear and are bonded to the instrument tip by ultra-violet adhesive.

18. An electrosurgical resector tool according to any preceding claim,
wherein the energy conveying structure comprises a coaxial transmission line extending in a longitudinal direction through the lumen, and wherein the coaxial transmission line comprises an inner conductor separated from an outer conductor by a dielectric material, and
wherein the inner conductor is connected to one of the first electrode and the second electrode and the outer conductor is connected to the other of the first electrode and the second electrode for delivery of the EM energy from the instrument tip.

19. An electrosurgical resector tool according to any preceding claim, wherein the energy conveying structure is for carrying radiofrequency (RF) electromagnetic (EM) energy and microwave EM energy, and
wherein the first electrode and the second electrode are operable:
as active and return electrodes for delivering RF energy conveyed from the energy conveying structure; and
a microwave field emitting structure for delivering microwave energy conveyed from the energy conveying structure.

20. An electrosurgical apparatus comprising:
an electrosurgical generator for supplying electromagnetic (EM) energy;
a surgical scoping device having an instrument cord for insertion into a patient's body, the instrument cord having an instrument channel extending therethrough;
an electrosurgical resector tool according to any preceding claim inserted through the instrument channel of the surgical scoping device.

21. An electrosurgical apparatus according to claim 20, when dependent on claim 19, wherein the electrosurgical generator is capable of supplying radiofrequency (RF) EM energy and microwave EM energy.

22. An electrosurgical resector tool comprising:
a shaft defining a lumen;
an energy conveying structure for carrying electromagnetic (EM) energy through the lumen of the shaft;
an instrument tip mounted at a distal end of the shaft, wherein the instrument tip comprises:
a static portion comprising a first blade element; and
a movable portion comprising a second blade element, wherein the movable portion is movable relative to the static portion between a closed position in which the first blade element and second blade element lie alongside each other to an open position in which the second blade element is spaced from the first blade element by a gap for receiving biological tissue;
a first electrode, a second electrode and a planar dielectric body, the first and second electrodes being spaced apart and electrically isolated from each other by the planar dielectric body, and wherein the first electrode and the second electrode are connected to the energy conveying structure for delivery of the EM energy from the instrument tip;
an actuator for controlling relative movement between the movable portion and the static portion, the actuator comprising a control rod slidably mounted in the shaft, the control rod having an attachment feature engaged with the movable portion, whereby longitudinal movement of the control rod in the shaft causes movement of the movable portion relative to the static portion; and
a first tube, a second tube and a third tube, wherein the first tube surrounds the control rod except a distal end region of the control rod, wherein the second tube surrounds the distal end region of the control rod except the attachment feature of the control rod, and the second tube protrudes proximally into the first tube to define an overlap region where the first tube overlaps the second tube, and wherein the third tube surrounds the overlap region and a proximal end region of the static portion.

23. An electrosurgical resector tool according to claim 22, wherein the first, second and third tubes are substantially clear and are bonded to the instrument tip by ultra-violet adhesive.

24. An electrosurgical resector tool according to claim 22 or 23, wherein the instrument tip further comprises a travel limiting mechanism operable to limit a maximum extent of relative movement between the second blade element and the first blade element in the open position and/or the closed position.
